(19) United States
(12) Patent Application Publication
(10) Pub. No.: US 2012/0039847 A1
(43) Pub. Date: Feb. 16, 2012

(54) Hepatitis C Virus Inhibitors

(76) Inventor: Shu-Hai Zhao, Shanghai (CN)

(21) Appl. No.: 13/208,382

(22) Filed: Aug. 12, 2011

Related U.S. Application Data
(60) Provisional application No. 61/373,406, filed on Aug. 13, 2010.

(51) Int. Cl. A61K 31/4178; C07D 215/38; C12N 5/071; A61K 31/4709; A61K 38/21; A61P 31/14; C07D 403/14; C07D 403/04

(52) U.S. Cl. 424/85.4; 548/314.7; 546/171; 514/397; 514/314; 435/375

(57) ABSTRACT

The present invention provides compounds, compositions and methods for the treatment of hepatitis C virus (HCV) infection. Also disclosed are pharmaceutical compositions containing such compounds and methods for using these compounds in the treatment of HCV infection.

### CROSS-REFERENCE TO RELATED APPLICATIONS

This application is entitled to the benefit of U.S. provisional patent application Ser. No. 61/373,406 filed on Aug. 13, 2010, the disclosure of which is incorporated herein by reference.

### FIELD OF THE INVENTION

The present invention relates to antiviral compounds, compositions comprising the same and methods for using such compounds and compositions which are useful inhibitors of HCV.

### BACKGROUND OF THE INVENTION

HCV is a major human pathogen, infecting an estimated 170 million persons worldwide. A substantial fraction of these HCV infected individuals develop serious progressive liver disease such as chronic hepatitis, cirrhosis, liver failure and hepatocellular carcinoma. Chronic HCV infection is thus a major worldwide cause of liver-related premature mortality.

HCV is a positive-stranded RNA virus and are classified as a separate genus in the Flaviviridae family. All members of the Flaviviridae family have enveloped virions that contain a positive stranded RNA genome encoding all known virus-specific proteins via translation of a single, uninterrupted, open reading frame. Considerable heterogeneity is found within the nucleotide and encoded amino acid sequence throughout the HCV genome. At least six major genotypes have been characterized, and more than 50 subtypes have been described.

The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins. In the case of HCV, the generation of mature non-structural proteins (e.g., NS2, NS3, NS4A, NS4B, NS5A, and NS5B) is effected by two viral proteases. The first one is believed to be a metalloprotease and cleaves at the NS2-NS3 junction; the second one is a serine protease contained within the N-terminal region of NS3 (also referred to herein as NS3 protease) and mediates all the subsequent cleavages downstream of NS3, both in cis, at the NS3-NS4A cleavage site, and in trans, for the remaining NS4A-NS4B, NS4B-NS5A, NS5A-NS5B sites. The NS4A protein appears to serve multiple functions, acting as a cofactor for the NS3 protease and possibly assisting in the membrane localization of NS3 and other viral replicase components. The complex formation of the NS3 protein with NS4A seems necessary to the processing events, enhancing the proteolytic efficiency at all of the sites. The NS3 protein also exhibits nucleoside triphosphatase and RNA helicase activities. NS5B (also referred to herein as HCV polymerase) is a RNA-dependent RNA polymerase that is involved in the replication of HCV.

Compounds useful for treating HCV-infected patients are desired which selectively inhibit HCV viral replication.

Currently, one of the most effective HCV treatments uses a combination of α-interferon and ribavirin, leading to sustained efficacy in only about 40% of patients. Thus, a substantial fraction of patients do not have a sustained reduction in viral load. Moreover, the treatment is cumbersome and sometimes has debilitating and severe side effects and many patients do not durably respond to treatment.

Thus, there is a continuing need to develop effective therapeutics for treatment of HCV infection.

### SUMMARY OF THE INVENTION

Some aspects of the invention provide compounds of the formula I or a pharmaceutically

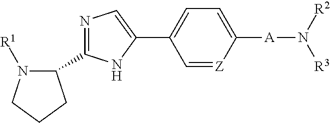

acceptable salt thereof. Such compounds and compositions comprising such a compound are useful in a wide variety of therapeutic applications including inhibiting hepatitis virus C and treating HCV infection. In compounds of Formula I, A is a moiety selected from the group consisting of optionally substituted moieties of the formulas:

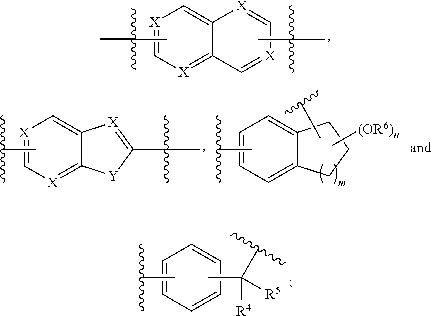

each of which is optionally substituted with C₁₋₆ alkyl; C₁₋₃ haloalkyl, C₁₋₆ alkoxy, halogen, hydroxy, carboxyl, C₁₋₆ alkoxycarbonyl, C₁₋₆ hydroxyalkyl, C₃₋₇ cycloalkyl, cyano or (CH₂)₀₋₃NR^{a}R^{b}.
m is 1 or 2.
n is 0 or 1.

Each X and Z is independently CH or N, provided that no more than two of X are N. Y is C(═O), O, S, or NR⁷.

R¹ is glycine or an aliphatic amino acid which optionally is N-acylated with a C₁₋₆ acyl, a benzoyl group or a C₁₋₆ alkoxycarbonyl group.

### R² is Pro-R¹ or Ala-R¹.

R³ is hydrogen or C₁₋₆ alkyl.
R⁴ is hydrogen, C₁₋₆ alkyl, C₃₋₆ cycloalkyl, C₁₋₆ haloalkyl, C₁₋₆ hydroxyalkyl, c1-4 alkoxy-C₁₋₆ alkyl, or (CH₂)₀₋₃NR^{a}R^{b}.
R⁵ is hydrogen, C₁₋₆ alkyl, C₁₋₆ haloalkyl, C₁₋₆ hydroxyalkyl, C₁₋₃ alkoxy-C₁₋₆ alkyl, or (CH₂)₀₋₃NR^{a}R^{b}.
R⁶ is hydrogen, C₁₋₆ alkyl, C₁₋₆ haloalkyl, C₁₋₆ hydroxyalkyl, C₁₋₃ alkoxy-C₁₋₆ alkyl, cyano-C₁₋₃ alkyl, or a hydroxy protecting group.
R⁷ is hydrogen, C₁₋₆ alkyl, or nitrogen-protecting group.
R^{a} and R^{b} are independently hydrogen or C₁₋₆ alkyl.

Another aspect of the invention relates to composition comprising a compound of Formula I.

Still other aspects of the invention relate to methods for treating HCV infection and methods for inhibiting HCV.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “ [symbol] ” drawn through a bond each refer to the point of attachment of a functional group or other chemical moiety to the rest of the molecule of which it is a part. Thus, for example:

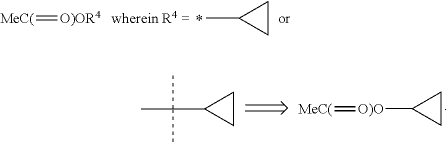

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

The term “alkyl” as used herein refers to a saturated linear monovalent hydrocarbon moiety of one to twelve, typically one to six, carbon atoms or a saturated branched monovalent hydrocarbon moiety of three to twelve, typically three to six, carbon atoms. Exemplary alkyl group include, but are not limited to, methyl, ethyl, n-propyl, 2-propyl, tert-butyl, pentyl, and the like.

The term “alkylene” as used herein refers to a saturated linear or branched divalent hydrocarbon moiety of one to twelve, typically one to six, carbon atoms or a branched saturated divalent hydrocarbon moiety of three to twelve, typically three to six, carbon atoms. Exemplary alkylene groups include, but are not limited to, methylene, ethylene, propylene, butylene, pentylene, and the like.

The term “amino acid residue” as used herein refers to an amino acid moiety that is linked to a functional group of another molecule including another amino acid, typically with a loss of a water molecule and becoming chemically bonded to a functional group of another molecule. The term “aliphatic amino acid” as used herein refers to a C₁₋₁₀ carboxylic acid substituted with an amino group at the 2-position. The symbol “Pro-R¹” or “Ala-R¹” as used herein refers to a dipeptide in which the C-terminus is proline or alanine respective and the N-terminus is an aliphatic amino acid as described herein.

The term “aryl” as used herein refers to a monovalent mono- or bicyclic aromatic hydrocarbon moiety of 6 to 10 ring atoms which is optionally substituted with one or more, typically one, two, or three substituents within the ring structure with C₁₋₆ alkyl; C₁₋₃ haloalkyl, C₁₋₆ alkoxy, halogen, hydroxy, carboxyl, C₁₋₆ alkoxycarbonyl, C₁₋₆ hydroxyalkyl, cyano or (CH₂)₀₋₃NR^{a}R^{b}. When two or more substituents are present in an aryl group, each substituent is independently selected.

The term “aralkyl” as used herein refers to a moiety of the formula —R^{c}R^{d} where R^{c} is an alkylene group and R^{d} is an aryl group as defined herein. Exemplary aralkyl groups include, but are not limited to, benzyl, phenylethyl, 3-(3-chlorophenyl)-2-methylpentyl, and the like.

The term “chiral center” (i.e., stereochemical center, stereocenter, or stereogenic center) as used herein refers to an asymmetrically substituted atom, e.g., a carbon atom to which four different groups are attached. The ultimate criterion of a chiral center, however, is nonsuperimposability of its mirror image.

The term “cycloalkyl” as used herein refers to a non-aromatic, typically saturated, monovalent mono- or bicyclic hydrocarbon moiety of three to ten ring carbons. The cycloalkyl can be optionally substituted with one or more, typically one, two, or three, substituents within the ring structure. When two or more substituents are present in a cycloalkyl group, each substituent is independently selected. Typical substituents for cycloalkyl group include with C₁₋₆ alkyl; C₁₋₃ haloalkyl, C₁₋₆ alkoxy, halogen, hydroxy, carboxyl, C₁₋₆ alkoxycarbonyl, C₁₋₆ hydroxyalkyl, cyano or (CH₂)₀₋₃NR^{a}R^{b}. Exemplary cycloalkyl includes, but are not limited to, cyclopropyl, cyclopentyl and cyclohexyl.

The term “cycloalkylalkyl” as used herein refers to a moiety of the formula —R^{e}R^{f} where R^{e} is an alkylene group and R^{f} is a cycloalkyl group as defined herein.

The term “enantiomeric excess” as used herein refers to the difference between the amount of enantiomers. The percentage of enantiomeric excess (% ee) can be calculated by subtracting the percentage of one enantiomer from the percentage of the other enantiomer. For example, if the % ee of (R)-enantiomer is 99% and % ee of (S)-enantiomer is 1%, the % ee of (R)-isomer is 99%-1% or 98%.

The terms “halo,” “halogen” and “halide” as used herein are used interchangeably herein and refer to fluoro, chloro, bromo, or iodo.

The term “haloalkyl” as used herein refers to an alkyl group as defined herein in which one or more hydrogen atom is replaced by same or different halo atoms. The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like.

The term “hydroxyalkyl” as used herein refers to an alkyl group as defined herein in which at least one hydrogen is replaced with a hydroxyl group (—OH).

The term “alkoxyalkyl” as used herein refers to an alkyl group as defined herein in which at least one hydrogen is replaced with an alkoxy group (—OR, where R is alkyl).

The term “cyanoalkyl” as used herein refers to an alkyl group as defined herein in which at least one hydrogen is replaced with a cyano group (—CN).

The term “acyl” [or “alkanoyl”] as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl [or “alkanoyl”] refers to a group —C(═O)R contain 1 to 6 carbon atoms. The C₁ acyl [or “alkanoyl”] group is the formyl group wherein R═H and a C₆ acyl group refers to hexanoyl when the alkyl chain is unbranched. The term “arylcarbonyl” or “aroyl” as used herein means a group of formula C(═O)R wherein R is an aryl group; the term “benzoyl” as used herein an “arylcarbonyl” or “aroyl” group wherein R is phenyl.

The terms “alkoxycarbonyl” and “aryloxycarbonyl” as used herein denotes a group of formula —C(═O)OR wherein R is alkyl or aryl respectively and alkyl and aryl are as defined herein.

The term “leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like.

A “pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The term “protecting group” as used herein refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers, allyl ethers, and —C(═O)NR^{a}R^{b}, where each of R^{a} and R^{b} is independently hydrogen or alkyl. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

The term “corresponding protecting group” as used herein means an appropriate protecting group corresponding to the heteroatom (i.e., N, O, P or S) to which it is attached.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) inhibiting the disease, i.e., arresting or reducing the development of the disease or its clinical symptoms; or (2) relieving the disease, i.e., causing regression of the disease or its clinical symptoms.

When describing a chemical reaction, the terms “treating”, “contacting” and “reacting” are used interchangeably herein, and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Compounds of the Invention

Some aspects of the invention provide compounds of the formula:

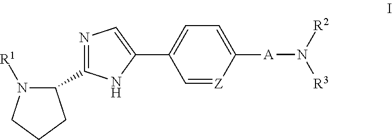

or a pharmaceutically acceptable salt thereof. In compounds of Formula (I), A is a moiety selected from the group consisting of optionally substituted moieties of the formulas:

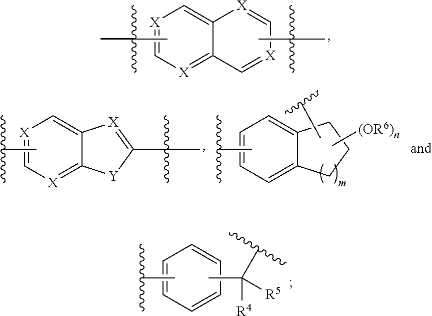

each of which is optionally substituted with C₁₋₆ alkyl; C₁₋₃ haloalkyl, C₁₋₆ alkoxy, halogen, hydroxy, carboxyl, C₁₋₆ alkoxycarbonyl, C₁₋₆ hydroxyalkyl, cyano or (CH₂)₀₋₃NR^{a}R^{b};
   m is 1 or 2.
   n is 0 or 1.

Each X and Z is independently CH or N, provided that no more than two of X is N. Y is C(═O), O, S, or NR⁷.

R¹ is glycine or an aliphatic amino acid which is optionally is N-acylated with a C₁₋₆ acyl, a benzoyl group or a C₁₋₆ alkoxycarbonyl group.

### R² is Pro-R¹ or Ala-R¹.

R³ is hydrogen or C₁₋₆ alkyl.
R⁴ is hydrogen, C₁₋₆ alkyl, C₃₋₆ cycloalkyl, C₁₋₆ haloalkyl, C₁₋₆ hydroxyalkyl, c1-4 alkoxy-C₁₋₆ alkyl, or (CH₂)₀₋₃NR^{a}R^{b}.
R⁵ is hydrogen, C₁₋₆ alkyl, C₁₋₆ haloalkyl, C₁₋₆ hydroxyalkyl, C₁₋₃ alkoxy-C₁₋₆ alkyl, or (CH₂)₀₋₃NR^{a}R^{b}.
R⁶ is hydrogen, C₁₋₆ alkyl, C₁₋₆ haloalkyl, C₁₋₆ hydroxyalkyl, C₁₋₃ alkoxy-C₁₋₆ alkyl, cyano-C₁₋₃ alkyl, or a hydroxy protecting group.
R⁷ is hydrogen, C₁₋₆ alkyl, or nitrogen-protecting group.
R^{a} and R^{b} are independently hydrogen or C₁₋₆ alkyl.

In some embodiments, the amino acid residue is an (L)-amino acid residue. Exemplary N-acyl groups include C₁₋₆ acyl, a benzoyl group or a C₁₋₆ alkoxycarbonyl group groups that are known to one skilled in the art. It should be appreciated that when the amino acid residue includes additional function group, for example, hydroxy group in serine, or a thiol group in cysteine, such a functional group can also be protected with a corresponding protecting group, which are well known to one skilled in the art.

In an embodiment of the present invention R¹ amino acid residue includes valine, proline, leucine, isoleucine or another C₁₋₁₀ aliphatic amino acid as defined above. Thus the definition encompasses both natural and non-natural amino acids. In some instances R¹ is valine. In another embodiment R² is Pro-R¹ or Ala-R¹. In another embodiment R² is Pro-R¹ or Ala-R¹. In yet another embodiment R² is Pro-Val. It should be appreciated that R¹ and R² are optionally N-acyl amino acids protected amino acid residues. Furthermore, the amino acid can be either (D)- or (L)-amino acid.

Still in other embodiments, R³ is hydrogen.

Yet in some embodiments, Z is CH.

Other embodiments of the invention include compounds of Formula I, where A is a moiety selected from the group consisting of:

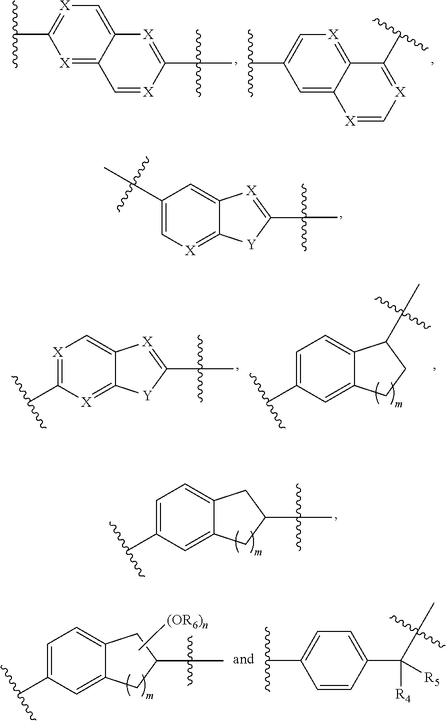

where X, Y, R⁴, R⁵, R⁶, m, and n are those defined herein.

In other embodiments A is a moiety selected from the group consisting of:

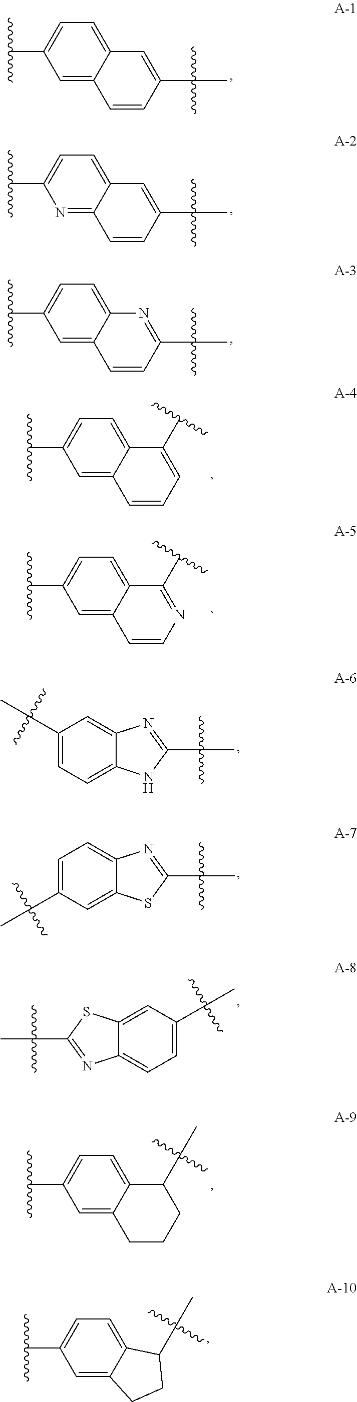

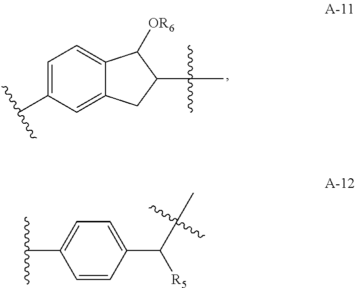

where R⁵ and R⁶ are those defined herein.

It should be appreciated that combinations of the different groups described herein can form other embodiments. In this manner, a variety of different compounds are embodied within the present invention. For example, in one particular embodiment, A is a moiety of the formula:

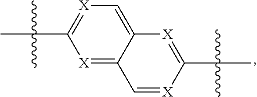

X's and Z are CH, R¹ is Val-NHBoc, R² is -Pro-Val-NHBoc, and R³ is hydrogen.

In one embodiment of the invention A is A-1 and R¹ and R² are as defined hereinabove. In another embodiment of the present invention A is A-2 and R¹ and R² are as defined hereinabove. In another embodiment of the present invention A is A-3 and R¹ and R² are as defined hereinabove. In another embodiment of the present invention A is A-4 and R¹ and R² are as defined hereinabove. In another embodiment of the present invention A is A-5 and R¹ and R² are as defined hereinabove. In another embodiment of the present invention A is A-6 and R¹ and R² are as defined hereinabove. In another embodiment of the present invention A is A-7 and R¹ and R² are as defined hereinabove. In another embodiment of the present invention A is A-8 and R¹ and R² are as defined hereinabove. In another embodiment of the present invention A is A-9 and R¹ and R² are as defined hereinabove. In another embodiment of the present invention A is A-10 and R¹ and R² are as defined hereinabove. In another embodiment of the present invention A is A-12, R⁵ is H and R¹ and R² are as defined hereinabove.

Representative compounds in accordance with the invention are shown in Table I.

TABLE I

| Cpd. | | IC₅₀ | |
|---|---|---|---|
| No. | STRUCTURE/NAME | nm¹ | MS |
| I-1 | 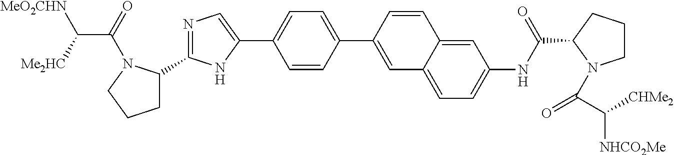 | 0.041 | 766.6 |
| | ((S)-1-{(S)-2-[6-(4-{2-[(S)-1-((S)-2-Methoxycarbonylamino-3- | | |
| | methyl-butyryl)-pyrrolidin-2-yl]-3H-imidazol-4-yl}-phenyl)- | | |
| | naphthalen-2-ylcarbamoyl]-pyrrolidine-1-carbonyl}-2-methyl-propyl)- | | |
| | carbamic acid methyl ester | | |
| I-2 | 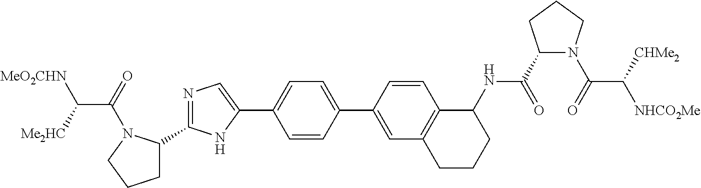 | 4.0 | 770.6 |
| | [(S)-1-((S)-2-{5-[4-(5-{[(S)-1-((S)-2-Methoxycarbonylamino-3- | | |
| | methyl-butyryl)-pyrrolidine-2-carbonyl]-amino}-5,6,7,8-tetrahydro- | | |
| | naphthalen-2-yl)-phenyl]-1H-imidazol-2-yl}-pyrrolidine-1-carbonyl)- | | |
| | 2-methyl-propyl]-carbamic acid methyl ester | | |
| I-3 | 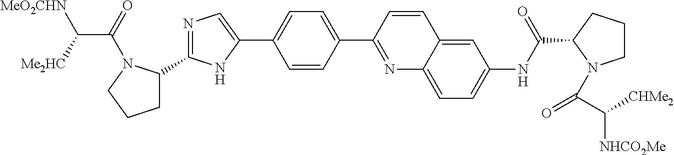 | 0.413 | 767.4 |
| | ((S)-1-{(S)-2-[2-(4-{2-[(S)-1-((S)-2-Methoxycarbonylamino-3- | | |
| | methyl-butyryl)-pyrrolidin-2-yl]-3H-imidazol-4-yl}-phenyl)-quinolin- | | |
| | 6-ylcarbamoyl]-pyrrolidine-1-carbonyl}-2-methyl-propyl)-carbamic | | |
| | acid methyl ester | | |
| I-4 | 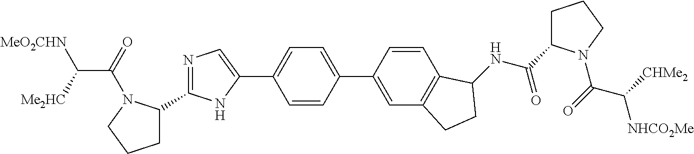 | 0.31 | 756.8 |
| | [(S)-1-((S)-2-{5-[4-(1-{[(S)-1-((S)-2-Methoxycarbonylamino-3- | | |
| | methyl-butyryl)-pyrrolidine-2-carbonyl]-amino}-indan-5-yl)-phenyl]- | | |
| | 1H-imidazol-2-yl}-pyrrolidine-1-carbonyl)-2-methyl-propyl]-carbamic | | |
| | acid methyl ester | | |
| I-5 | 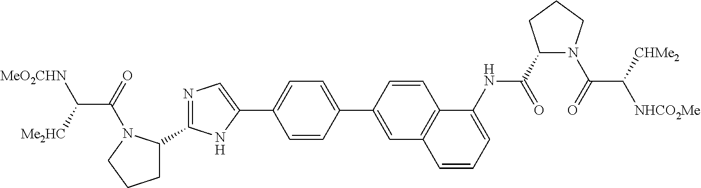 | 0.2 | 766.6 |
| | [(S)-1-((S)-2-{5-[4-(5-{[(S)-1-((S)-2-Methoxycarbonylamino-3- | | |
| | methyl-butyryl)-pyrrolidine-2-carbonyl]-amino}-naphthalen-2-yl)- | | |
| | phenyl]-1H-imidazol-2-yl}-pyrrolidine-1-carbonyl)-2-methyl-propyl]- | | |
| | carbamic acid methyl ester | | |
| I-6 | 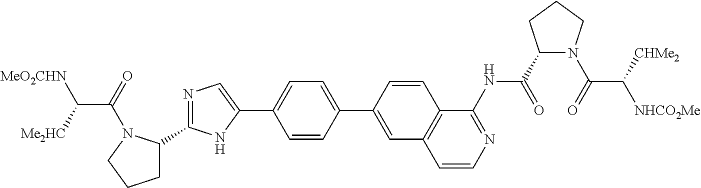 | 13.9 | 767.6 |
| | [(S)-1-((S)-2-{5-[4-(1-{[(S)-1-((S)-2-Methoxycarbonylamino-3- | | |
| | methyl-butyryl)-pyrrolidine-2-carbonyl]-amino}-isoquinolin-6-yl)- | | |
| | phenyl]-1H-imidazol-2-yl}-pyrrolidine-1-carbonyl)-2-methyl-propyl]- | | |
| | carbamic acid methyl ester | | |
| I-7 | 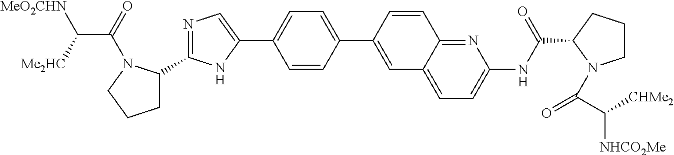 | 0.15 | 767.5 |
| | ((S)-1-{(S)-2-[6-(4-{2-[(S)-1-((S)-2-Methoxycarbonylamino-3- | | |
| | methyl-butyryl)-pyrrolidin-2-yl]-3H-imidazol-4-yl}-phenyl)-quinolin- | | |
| | 2-ylcarbamoyl]-pyrrolidine-1-carbonyl}-2-methyl-propyl)-carbamic | | |
| | acid methyl ester | | |
| I-8 | 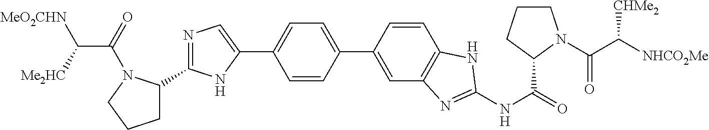 | 0.37 | 756.8 |
| | [(S)-1-((S)-2-{5-[4-(2-{[(S)-1-((S)-2-Methoxycarbonylamino-3- | | |
| | methyl-butyryl)-pyrrolidine-2-carbonyl]-amino}-1H-benzoimidazol-5- | | |
| | yl)-phenyl]-1H-imidazol-2-yl}-pyrrolidine-1-carbonyl)-2-methyl- | | |
| | propyl]-carbamic acid methyl ester | | |
| I-9 | 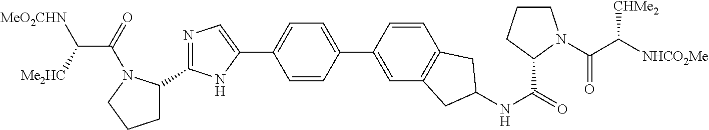 | 0.43 | 756.6 |
| | ((S)-1-{(S)-2-[5-(4-{2-[(S)-1-((S)-2-Methoxycarbonylamino-3- | | |
| | methyl-butyryl)-pyrrolidin-2-yl]-3H-imidazol-4-yl}-phenyl)-indan-2- | | |
| | ylcarbamoyl]-pyrrolidine-1-carbonyl}-2-methyl-propyl)-carbamic acid | | |
| | methyl ester | | |
| I-10 | 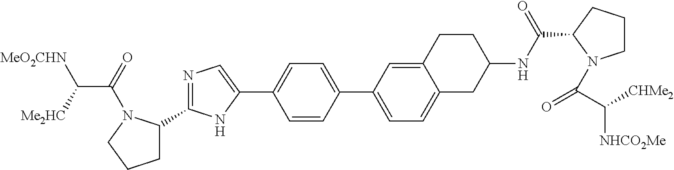 | 1.2 | 770.8 |
| | ((S)-1-{(S)-2-[6-(4-{2-[(S)-1-((S)-2-Methoxycarbonylamino-3- | | |
| | methyl-butyryl)-pyrrolidin-2-yl]-3H-imidazol-4-yl}-phenyl)-1,2,3,4- | | |
| | tetrahydro-naphthalen-2-ylcarbamoyl]-pyrrolidine-1-carbonyl}-2- | | |
| | methyl-propyl)-carbamic acid methyl ester | | |
| I-11 | 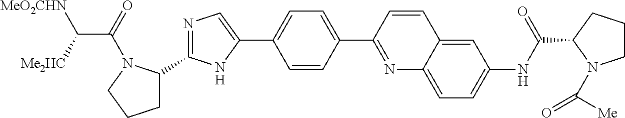 | 0.63 | 652 |
| | ((S)-1-{(S)-2-[5-(4-{6-[((S)-1-Acetyl-pyrrolidine-2-carbonyl)-amino]- | | |
| | quinolin-2-yl}-phenyl)-1H-imidazol-2-yl]-pyrrolidine-1-carbonyl}-2- | | |
| | methyl-propyl)-carbamic acid methyl ester | | |
| I-12 | 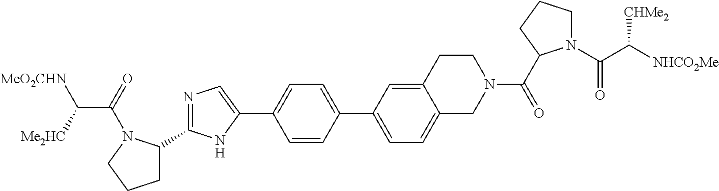 | 2.06 | 756.4 |
| | ((S)-1-{(S)-2-[5-(4-{2-[(S)-1-((S)-2-Methoxycarbonylamino-3- | | |
| | methyl-butyryl)-pyrrolidine-2-carbonyl]-1,2,3,4-tetrahydro- | | |
| | isoquinolin-6-yl}-phenyl)-1H-imidazol-2-yl]-pyrrolidine-1-carbonyl}- | | |
| | 2-methyl-propyl)-carbamic acid methyl ester | | |
| I-13 | 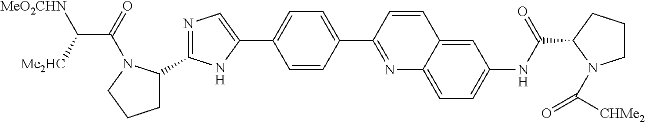 | 1.06 | 680.5 |
| | ((S)-1-{(S)-2-[5-(4-{6-[((S)-1-Isobutyryl-pyrrolidine-2-carbonyl)- | | |
| | amino]-quinolin-2-yl}-phenyl)-1H-imidazol-2-yl]-pyrrolidine-1- | | |
| | carbonyl}-2-methyl-propyl)-carbamic acid methyl ester | | |
| I-14 | 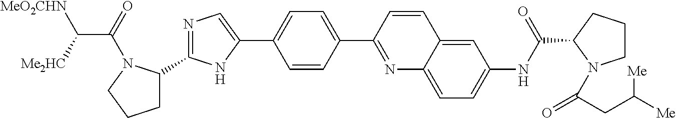 | 1.7 | 694.5 |
| | [(S)-2-Methyl-1-((S)-2-{5-[4-(6-{[(S)-1-(3-methyl-butyryl)- | | |
| | pyrrolidine-2-carbonyl]-amino}-quinolin-2-yl)-phenyl]-1H-imidazol- | | |
| | 2-yl}-pyrrolidine-1-carbonyl)-propyl]-carbamic acid methyl ester | | |
| I-15 | 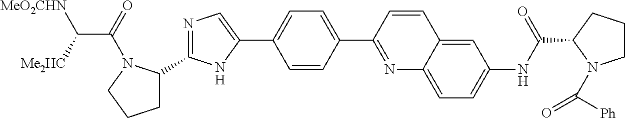 | 1.4 | 714.5 |
| | ((S)-1-{(S)-2-[5-(4-{6-[((S)-1-Benzoyl-pyrrolidine-2-carbonyl)- | | |
| | amino]-quinolin-2-yl}-phenyl)-1H-imidazol-2-yl]-pyrrolidine-1- | | |
| | carbonyl}-2-methyl-propyl)-carbamic acid methyl ester | | |
| I-16 | 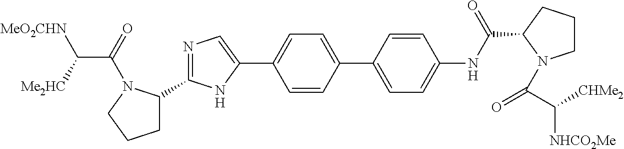 | 1.23 | 730.2 |
| | ((S)-1-{(S)-2-[(4′-{2-[(S)-1-((S)-2-Methoxycarbonylamino-3-methyl- | | |
| | butyryl)-pyrrolidin-2-yl]-3H-imidazol-4-yl}-biphenyl-4-ylmethyl)- | | |
| | carbamoyl]-pyrrolidine-1-carbonyl}-2-methyl-propyl)-carbamic acid | | |
| | methyl ester | | |

¹IC₅₀ nM HCV replicon assay-see example 3

The compounds represented in TABLE II illustrate other compounds encompassed with the present invention.

TABLE II

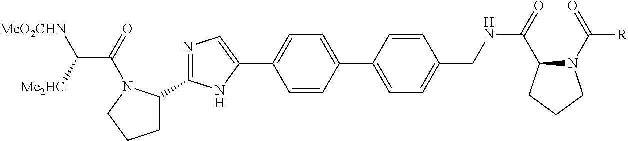

R = —CF₃, —CHF₂, —CH₂OR^{a}, —CONR^{b}R^{c}, where each of R^{a}, R^{b},
and R^{c} is independently hydrogen or alkyl.

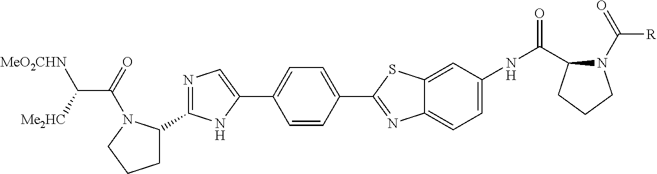

R = —CF₃, —CHF₂, —CH₂OR^{a}, —OR^{a}, —CONR^{b}R^{c}, where each of
R^{a}, R^{b}, and R^{c} is independently hydrogen or alkyl.

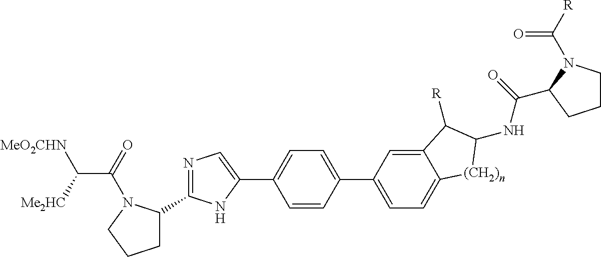

n = 1 or 2, R = —CF₃, —CHF₂, —CH₂OR^{a}, —OR^{a}, —CONR^{b}R^{c},
where each of R^{a}, R^{b}, and R^{c} is independently hydrogen or
alkyl.

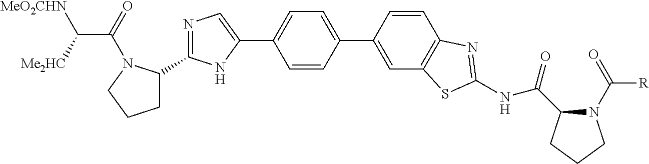

R = —CF₃, —CHF₂, —CH₂OR^{a}, —OR^{a}, —CONR^{b}R^{c}, where each of
R^{a}, R^{b}, and R^{c} is independently hydrogen or alkyl.

Another aspect of the invention provides a composition comprising a therapeutically effective amount of at least one compound of Formula I and a pharmaceutically acceptable carrier.

Yet another aspect of the invention provides a method for treating HCV in a subject comprising administering to the subject a therapeutically effective amount of a compound of Formula I.

Another aspect of the present invention provides a method for producing a compound of Formula I.

### Synthesis

Compounds of the invention can be made by a variety of methods depicted in the illustrative synthetic reactions described below in the Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

### Utility

The compounds of the invention have a variety of biological properties including antiviral activities. Therefore, they can be used in a variety of application including as a treatment for HCV infection.

Typically, a pharmaceutically or therapeutically effective amount of the composition is administered or delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. The subject can be administered as many doses as is required to reduce and/or alleviate the signs, symptoms or causes of the disorder in question, or bring about any other desired alteration of a biological system. One of ordinary skill in the art of treating such diseases can readily, without undue experimentation and in reliance upon personal knowledge and the disclosure of this application, ascertain a therapeutically effective amount of the compounds of this invention for a given disease.

The compounds of the invention and their isomeric forms and pharmaceutically acceptable salts thereof are useful in treating and preventing HCV infection alone or when used in combination with other compounds targeting viral or cellular elements or functions involved in the HCV lifecycle. Classes of compounds useful in the invention include, without limitation, all classes of HCV antivirals. For combination therapies, mechanistic classes of agents that can be useful when combined with the compounds of the invention include, for example, nucleoside and non-nucleoside inhibitors of the HCV polymerase, protease inhibitors, helicase inhibitors, NS4B inhibitors and medicinal agents that functionally inhibit the internal ribosomal entry site (IRES) and other medicaments that inhibit HCV cell attachment or virus entry, HCV RNA translation, HCV RNA transcription, replication or HCV maturation, assembly or virus release. Specific compounds in these classes and useful in the invention include, but are not limited to, macrocyclic, heterocyclic and linear HCV protease inhibitors such as telaprevir (VX-950), boceprevir (SCH-503034), narlaprevir (SCH-9005 18), ITMN-191 (R-7227), TMC-435350 (a.k.a. TMC-435), MK-7009, BI-201335, BI-2061 (ciluprevir), BMS-650032, ACH-1625, ACH-1095 (HCV NS4A protease co-factor inhibitor), VX-500, VX-8 13, PHX-1766, PHX2054, IDX-136, IDX-3 16, ABT-450 EP-0 13420 (and congeners) and VBY-376; the Nucleosidic HCV polymerase (replicase) inhibitors useful in the invention include, but are not limited to, R7128, PSI-785 1, IDX-184, IDX-102, R1479, UNX-08 189, PSI-6130, PSI-938 and PSI-879 and various other nucleoside and nucleotide analogs and HCV inhibitors including (but not limited to) those derived as 2′-C-methyl modified nucleos(t)ides, 4′-aza modified nucleos(t)ides, and 7′-deaza modified nucleos(t)ides. Non-nuclosidic HCV polymerase (replicase) inhibitors useful in the invention, include, but are not limited to, HCV-796, HCV-371, VCH-759, VCH-916, VCH-222, ANA-598, MK-3281, ABT-333, ABT-072, PF-00868554, BI-207127, GS-9190, A-837093, JKT-109, GL-59728 and GL-60667.

In addition, compounds of the invention can be used in combination with cyclophyllin and immunophyllin antagonists (e.g., without limitation, DEBIO compounds, NM-811 as well as cyclosporine and its derivatives), kinase inhibitors, inhibitors of heat shock proteins (e.g., HSP90 and HSP70), other immunomodulatory agents that can include, without limitation, interferons (-alpha, -beta, -omega, -gamma, -lambda or synthetic) such as Intron A, Roferon-A, Canferon-A300, Advaferon, Infergen, Humoferon, Sumiferon MP, Alfaferone, IFN-β, Feron and the like; polyethylene glycol derivatized (pegylated) interferon compounds, such as PEG interferon-α-2a (Pegasys), PEG interferon-α-2b (PEGlntron), pegylated IFN-α-con1 and the like; long acting formulations and derivatizations of interferon compounds such as the albumin-fused interferon, Albuferon, Locteron, and the like; interferons with various types of controlled delivery systems (e.g., ITCA-638, omega-interferon delivered by the DUROS subcutaneous delivery system); compounds that stimulate the synthesis of interferon in cells, such as resiquimod and the like; interleukins; compounds that enhance the development of type 1 helper T cell response, such as SCV-07 and the like; TOLL-like receptor agonists such as CpG-10101 (actilon), isotorabine, ANA773 and the like; thymosin α-1; ANA-245 and ANA-246; histamine dihydrochloride; propagermanium; tetrachlorodecaoxide; ampligen; IMP-321; KRN-7000; antibodies, such as civacir, XTL-6865 and the like and prophylactic and therapeutic vaccines such as InnoVac C, HCV E1E2/MF59 and the like. In addition, any of the above-described methods involving administering an NS5A inhibitor, a Type I interferon receptor agonist (e.g., an IFN-α) and a Type II interferon receptor agonist (e.g., an IFN-γ) can be augmented by administration of an effective amount of a TNF-α antagonist. Exemplary, non-limiting TNF-α antagonists that are suitable for use in such combination therapies include ENBREL, REMICADE, and HUMIRA.

In addition, compounds of the invention can be used in combination with antiprotozoans and other antivirals thought to be effective in the treatment of HCV infection such as, without limitation, the prodrug nitazoxanide. Nitazoxanide can be used as an agent in combination with the compounds disclosed in this invention as well as in combination with other agents useful in treating HCV infection such as peginterferon α-2a and ribavarin

Compounds of the invention can also be used with alternative forms of interferons and pegylated interferons, ribavirin or its analogs (e.g., tarabavarin, levoviron), microRNA, small interfering RNA compounds (e.g., SIRPLEX-140-N and the like), nucleotide or nucleoside analogs, immunoglobulins, hepatoprotectants, anti-inflammatory agents and other inhibitors of NS5A. Inhibitors of other targets in the HCV lifecycle include NS3 helicase inhibitors; NS4A co-factor inhibitors; antisense oligonucleotide inhibitors, such as ISIS-14803, AVI-4065 and the like; vector-encoded short hairpin RNA (shRNA); HCV specific ribozymes such as heptazyme, RPI, 13919 and the like; entry inhibitors such as HepeX-C, HuMax-HepC and the like; alpha glucosidase inhibitors such as celgosivir, UT-231B and the like; KPE-02003002 and BIVN 401 and IMPDH inhibitors. Other illustrative HCV inhibitor compounds include those disclosed in the following publications: U.S. Pat. Nos. 5,807,876; 6,498,178; 6,344,465; and 6,054,472; PCT Patent Application Publication Nos. WO97/40028; WO98/4038 1; WO00/56331, WO02/04425; WO03/007945; WO03/010141; WO03/000254; WO01/32153; WO00/06529; WO00/18231; WO00/10573; WO00/13708; WO01/85172; WO03/037893; WO03/037894; WO03/037895; WO02/100851; WO02/100846; WO99/01582; WO00/09543; WO02/18369; WO98/17679, WO00/056331; WO98/22496; WO99/07734; WO05/073216, WO05/073195 and WO08/021,927.

Additionally, combinations of, for example, ribavirin and interferon, may be administered as multiple combination therapy with at least one of the compounds of the invention. The present invention is not limited to the aforementioned classes or compounds and contemplates known and new compounds and combinations of biologically active agents. It is intended that combination therapies of the present invention include any chemically compatible combination of a compound of this inventive group with other compounds of the inventive group or other compounds outside of the inventive group, as long as the combination does not eliminate the anti-viral activity of the compound of this inventive group or the anti-viral activity of the pharmaceutical composition itself.

Combination therapy can be sequential, that is treatment with one agent first and then a second agent (for example, where each treatment comprises a different compound of the invention or where one treatment comprises a compound of the invention and the other comprises one or more biologically active agents) or it can be treatment with both agents at the same time (concurrently). Sequential therapy can include a reasonable time after the completion of the first therapy before beginning the second therapy. Treatment with both agents at the same time can be in the same daily dose or in separate doses. Combination therapy need not be limited to two agents and may include three or more agents. The dosages for both concurrent and sequential combination therapy will depend on absorption, distribution, metabolism and excretion rates of the components of the combination therapy as well as other factors known to one of skill in the art. Dosage values will also vary with the severity of the condition to be alleviated. It is to be further understood that for any particular subject, specific dosage regimens and schedules may be adjusted over time according to the individual's need and the judgment of the one skilled in the art administering or supervising the administration of the combination therapy.

### Administration and Pharmaceutical Composition

The present invention includes pharmaceutical compositions comprising at least one compound of the invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention are administered in a therapeutically effective amount by any of the accepted modes of administration for agents that serve similar utilities. Suitable dosage ranges are typically 1-500 mg daily, typically 1-100 mg daily, and often 1-30 mg daily, depending on numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases is typically able, without undue experimentation and in reliance upon personal knowledge and the disclosure of this application, to ascertain a therapeutically effective amount of the compounds of the invention.

Typically, compounds of the invention are administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. Typical manner of administration is generally oral using a convenient daily dosage regimen which can be adjusted according to the degree of affliction.

A compound or compounds of the invention, together with one or more conventional adjuvants, carriers, or diluents, can be placed into the form of pharmaceutical compositions and unit dosages. The pharmaceutical compositions and unit dosage forms can be comprised of conventional ingredients in conventional proportions, with or without additional active compounds or principles, and the unit dosage forms can contain any suitable effective amount of the active ingredient commensurate with the intended daily dosage range to be employed. The pharmaceutical compositions can be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention can be formulated in a wide variety of oral administration dosage forms. The pharmaceutical compositions and dosage forms can comprise a compound or compounds of the invention or pharmaceutically acceptable salts thereof as the active component. The pharmaceutically acceptable carriers can be either solid or liquid. Solid form preparations include powders, tablets, pills, capsules, cachets, suppositories, and dispersible granules. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions can be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

The compounds of the invention can also be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and can contain formulatory agents such as preserving, wetting, emulsifying or suspending, stabilizing and/or dispersing agents. Alternatively, the active ingredient can be in powder form, obtained by aseptic isolation of sterile solid or by lyophilization from solution for constitution before use with a suitable vehicle, e.g., sterile, pyrogen-free water.

The compounds of the invention can be formulated for topical administration to the epidermis as ointments, creams or lotions, or as a transdermal patch. Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention can be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention can also be formulated for vaginal administration. Pessaries, tampons, creams, gels, pastes, foams or sprays containing in addition to the active ingredient such carriers as are known in the art to be appropriate.

The compounds of the invention can be formulated for nasal administration. The solutions or suspensions are applied directly to the nasal cavity by conventional means, for example, with a dropper, pipette or spray. The formulations can be provided in a single or multidose form. In the latter case of a dropper or pipette, this can be achieved by the patient administering an appropriate, predetermined volume of the solution or suspension. In the case of a spray, this can be achieved for example by means of a metering atomizing spray pump.

The compounds of the invention can be formulated for aerosol administration, particularly to the respiratory tract and including intranasal administration. The compound will generally have a small particle size for example of the order of five (5) microns or less. Such a particle size can be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol can conveniently also contain a surfactant such as lecithin. The dose of drug can be controlled by a metered valve. Alternatively the active ingredients can be provided in a form of a dry powder, for example, a powder mix of the compound in a suitable powder base such as lactose, starch, starch derivatives such as hydroxypropylmethyl cellulose and polyvinylpyrrolidine (PVP). The powder carrier typically forms a gel in the nasal cavity. The powder composition can be presented in unit dose form, for example, in capsules or cartridges of e.g., gelatine or blister packs from which the powder can be administered by means of an inhaler.

When desired, formulations can be prepared with enteric coatings adapted for sustained or controlled release administration of the active ingredient. For example, the compounds of the invention can be formulated in transdermal or subcutaneous drug delivery devices. These delivery systems are advantageous when sustained release of the compound is necessary or desired and when patient compliance with a treatment regimen is crucial. Compounds in transdermal delivery systems are frequently attached to a skin-adhesive solid support. The compound of interest can also be combined with a penetration enhancer, e.g., Azone (1-dodecylazacycloheptan-2-one). Sustained release delivery systems can be inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are typically in unit dosage forms. In such form, the preparation is often subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa.

When it is possible that, for use in therapy, therapeutically effective amounts of a compound of Formula (I), as well as pharmaceutically acceptable salts thereof, can be administered as the raw chemical, it is possible to present the active ingredient as a pharmaceutical composition. Accordingly, the disclosure further provides pharmaceutical compositions, which include therapeutically effective mounts of compounds of Formula (I) or pharmaceutically acceptable salts thereof or a prodrug thereof, and one or more pharmaceutically acceptable carriers, diluents, or excipients. When applied to a combination, the term refers to combined amounts of the active ingredients that result in the therapeutic effect, whether administered in combination, serially, or simultaneously. The compounds of Formula (I) and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the disclosure there is also provided a process for the preparation of a pharmaceutical formulation including admixing a compound of Formula (I), or a pharmaceutically acceptable salt thereof, with one or more pharmaceutically acceptable carriers, diluents, or excipients.

When the compositions of this disclosure comprise a combination of a compound of the present disclosure and one or more additional therapeutic or prophylactic agent, both the compound and the additional agent are usually present at dosage levels of between about 10 to 150%, and more typically between about 10 and 80% of the dosage normally administered in a monotherapy regimen.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Compounds A-1 to A-5 shown below were prepared following the procedure disclosed in U.S. Patent Application Publication No. US20080299075, which is incorporated herein by reference in its entirety.

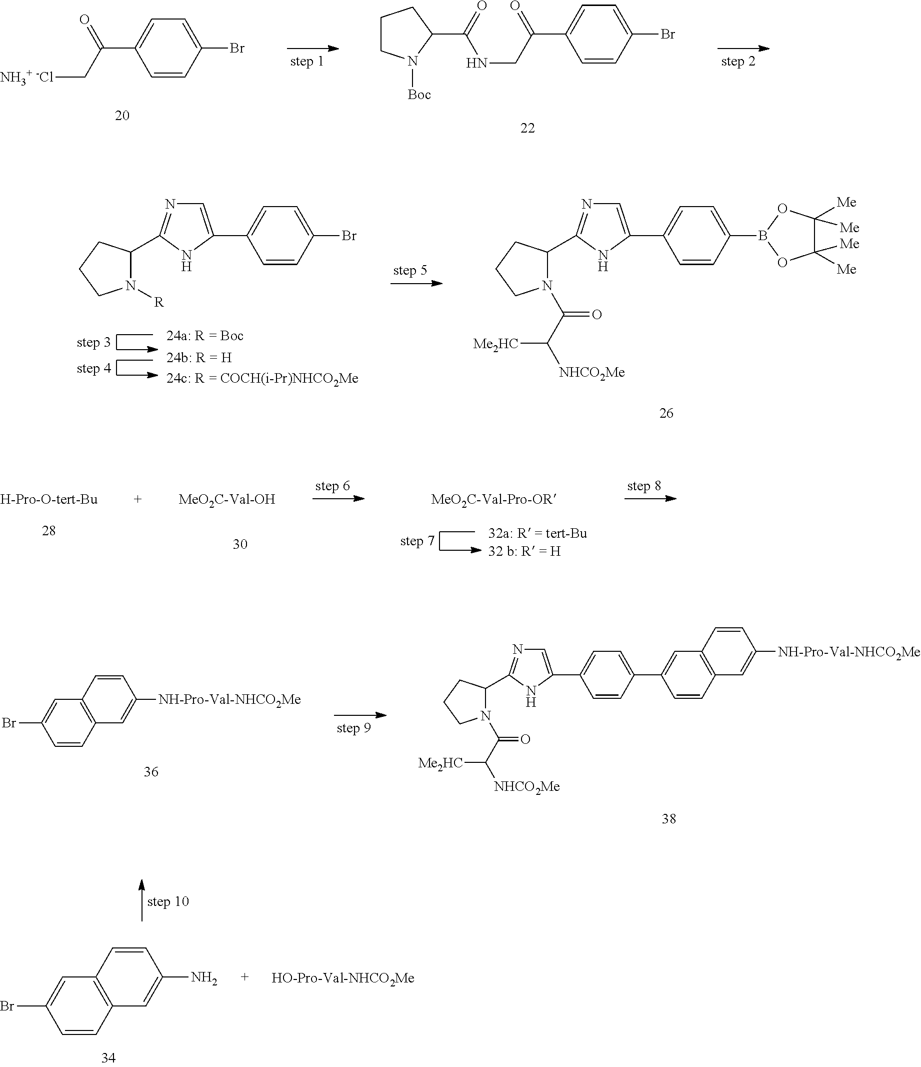

Acylation of amines to link peptides to other portions of the molecule (e.g., step 8) can be effected by preparing an activated carboxylic acid into a more reactive form such as an acid chloride or a symmetrical or mixed acid anhydride and reacting the activated derivative with an amine in an inert solvent such as DMF, DCM, THF, with or without water as a co-solvent, and the like at temperatures between 0° and 60° C. generally in the presence of a base such as Na₂CO₃, NaHCO₃, K₂CO₃, DIPEA, TEA or pyridine and the like to afford an amide. Carboxylic acids are converted into their acid chlorides using standard reagents well known to someone skilled in the art, such as thionyl chloride, oxalyl chloride, phosphoryl chloride and the like. Those reagents can be used in presence of bases such as DIPEA, TEA or pyridine.

Amide coupling to afford peptides (e.g., step 6) can carried out in situ using activated acids by known to those skilled in the art. These activated acids were reacted directly with the amines of to afford amides. Said activation of acids with those peptide coupling procedures can involve the use of an activating agent like EDC, DCC, benzotriazol-1-yloxy-tris(dimethylamino)phosphonium hexafluorophosphate (BOP), bromo-tris-pyrrolidinophosphonium hexafluorophosphate (PyBrOP), or 2-fluoro-1-methylpyridinium p-toluenesulphonate (Mukaiyama's reagent) and the like, optionally in the presence of modifiers such as HOBt, with or without a base such NMM, TEA or DIPEA in an inert solvent such as dimethylformamide (DMF) or dichloromethane at temperatures between 0° C. and 60° C. The reaction may alternatively be carried out in presence of O-(7-azabenzotriazol-1-yl)-N,N,N′,N′-tetramethyluronium hexafluorophosphate (HATU) or 1-hydroxy-7-azabenzotriazole (HOAt) and TEA or DIPEA in DMF, DCM or THF. Acylation of amines (J. March, supra pp. 417-425; H. G. Benz, Synthesis of Amides and Related Compounds in Comprehensive Organic Synthesis, E. Winterfeldt, ed., vol. 6, Pergamon Press, Oxford 1991 pp. 381-411; see R. C. Larock, Comprehensive Organic Transformations—A Guide to Functional Group Preparations, 1989, VCH Publishers Inc., New York; pp. 972-976) has been reviewed.

Aryl coupling (e.g., step 10) is typically carried out by palladium catalyzed coupling. The Suzuki reaction is particularly useful herein. The Suzuki reaction is a palladium-catalyzed coupling of a boronic acid with an aryl or vinyl halide or triflate. Typical catalysts bis-(tri-o-tolylphosphine)-palladium-(II)-chloride, tris-(dibenzylideneacetone)-dipalladium(0)/tris-o-tolylphosphine, tris-(dibenzylideneacetone)-dipalladium(0)/tris-(2-furyl)phosphan, tris-(dibenzylideneacetone)-dipalladium(1)/2,2′-bis-(diphenylphosphino)-1-,1′-binaphthyl, tetrakis-(triphenylphosphine)-palladium(0), 1,1′-bis-(diphenylphosphino)-ferrocene-palladium-dichloride or palladium-II-acetate/1,3-bis-(triphenylphosphino)-propane. The reaction id often carried out in the presence of a base such as sodium-tert.butoxide, bis-(trimethylsilyl)-lithium amide, potassium carbonate, caesium carbonate or triethylamine. The reaction can be carried out in a variety of organic solvents including toluene, THF, dioxane, 1,2-dichloroethane, DMF, DMSO and acetonitrile, aqueous solvents and under biphasic conditions. Reactions are typically run from about room temperature to about 150° C. Additives (e.g. CsF, KF, TlOH, NaOEt and KOH) frequently accelerate the coupling. Although there are numerous components in the Suzuki reaction including the palladium source, the ligand, additive solvent, temperature, etc., numerous protocols have been identified. Recently useful general conditions have been disclosed. R. Martin and S. L. Buchwald, Acc. Chem. Res. 2008 41(11):1461-73, A. F. Littke et al. J. Am. Chem. Soc. 2000 122:4020-4028 disclose conditions for Suzuki cross-coupling with arylboronic acids in high yield at RT utilizing Pd₂(dba)₃/P(tert-bu)₃ and conditions for cross-coupling of aryl- and vinyl triflates utilizing Pd(OAc)₂/P(C₆H₁₁)₃ at RT. J. P. Wolf et al. J. Am. Chem. Soc. 1999 121:9550-9561 disclose efficient condition for Suzuki cross-coupling utilizing Pd(OAc)₂/o-(di-tert-butylphosphino)biphenyl or o-(dicyclohexylyphosphino)biphenyl. One skilled in the art will be able to identify a satisfactory protocol without undue experimentation.

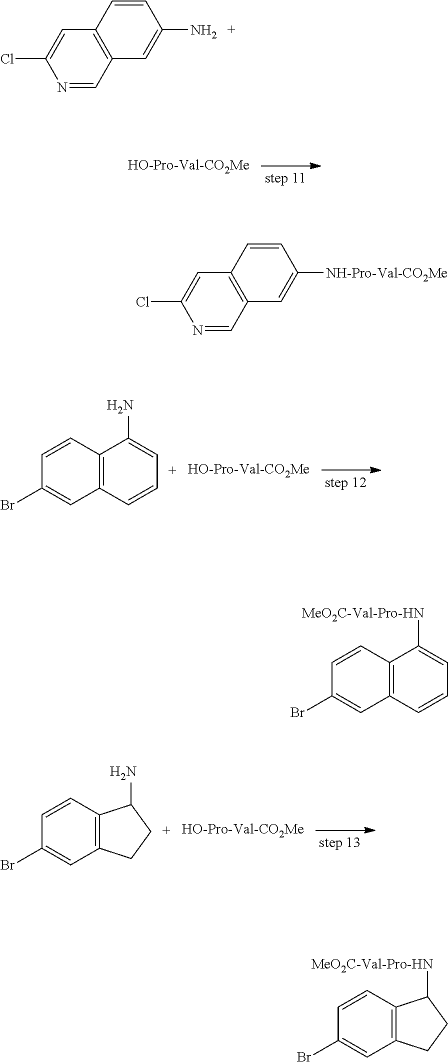

### Example 1

### {(S)-1-[(S)-2-(6-Bromo-naphthalen-2-ylcarbamoyl)-pyrrolidine-1-carbonyl]-2-methyl-propyl}-carbamic Acid Methyl Ester (20)

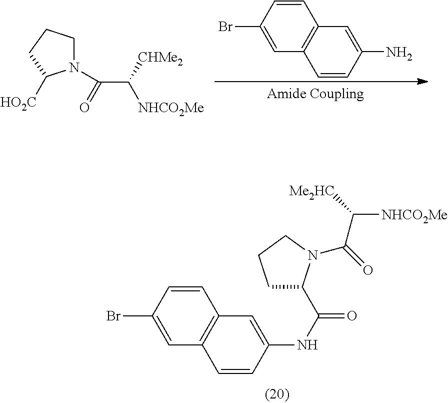

To a solution of 6-bromo-naphthalen-2-ylamine (326 mg, 1.47 mmol) and (S)-1-((S)-2-methoxy-carbonylamino-3-methyl-butyryl)-pyrrolidine-2-carboxylic acid (400 mg, 1.47 mmol) in 7 mL of DMSO, was added HATU (1.2 equiv) and DIPEA (1.2 equiv.). The mixture was stirred at room temperature for 16 h. The reaction was taken in EtOAC (50 mL) and washed with water and brine. The organic layer was dried, concentrated and chromatographed on silica gel using EtOAc/Hexanes as eluent to give the title compound (300 mg).

In a similar fashion the following compounds were prepared:
{(S)-1-[(S)-2-(2-Chloro-quinolin-6-ylcarbamoyl)-pyrrolidine-1-carbonyl]-2-methyl-propyl}-carbamic acid methyl ester;
{(S)-1-[(S)-2-(6-Bromo-naphthalen-1-ylcarbamoyl)-pyrrolidine-1-carbonyl]-2-methyl-propyl}-carbamic acid methyl ester;
{(S)-1-[(S)-2-(5-Bromo-indan-1-ylcarbamoyl)-pyrrolidine-1-carbonyl]-2-methyl-propyl}-carbamic acid methyl ester.

### Example 2

### ((S)-1-{(S)-2-[6-(4-{2-[(S)-1-((S)-2-Methoxycarbonylamino-3-methyl-butyryl)pyrrolidin-2-yl]-3H-imidazol-4-yl}-phenyl)-naphthalen-2-ylcarbamoyl]-pyrrolidine-1-carbonyl}-2-methyl-propyl)-carbamic Acid Methyl Ester

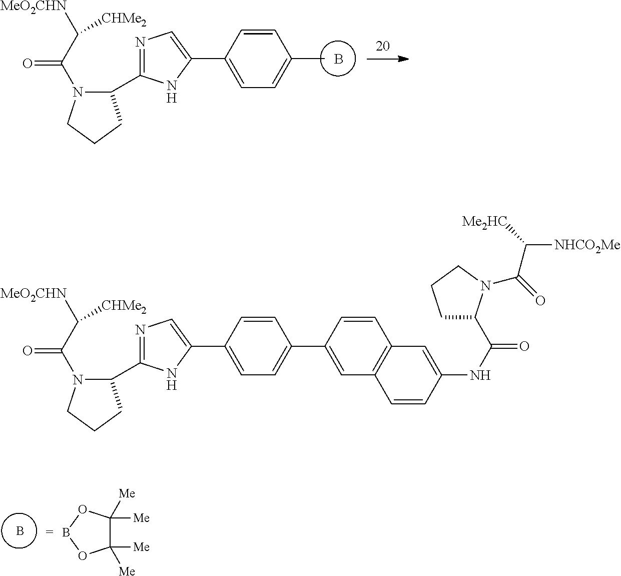

A mixture of [(S)-2-Methyl-1-((S)-2-{5-[4-(4,4,5,5 tetramethyl-[1,3,2]dioxaborolan-2-yl)-phenyl]-1H-imidazol-2-yl}-pyrrolidine-1-carbonyl)-propyl]-carbamic acid methyl ester (300 mg), 20 (1.1 equiv.) and NaHCO₃ (3.3 equiv) in DME (3 mL) and H2O (1 mL) was degassed with nitrogen. To the solution was added Pd(PPh3)4 (0.05 equiv.) and the reaction was heated at 80° C. for 18 h. The mixture was cooled to RT, diluted with EtOAc and washed with H2O. The organic layer was concentrated and purified by preparative HPLC to afford 30 mg of 22: MS: 766.6 (M+H)+.

Similarly, the following compounds were prepared:

[(S)-1-((S)-2-{5-[4-(1-{[(S)-1-((S)-2-Methoxycarbonylamino-3-methyl-butyryl)-pyrrolidine-2-carbonyl]-amino}-indan-5-yl)-phenyl]-1H-imidazol-2-yl}-pyrrolidine-1-carbonyl)-2-methyl-propyl]-carbamic acid methyl ester. MS: 756 (M+H)⁺1

((S)-1-{(S)-2-[6-(4-{2-[(S)-1-(S)-2-Methoxycarbonylamino-3-methyl-butyryl)-pyrrolidin-2-yl]-3H-imidazol-4-yl}-phenyl)-naphthalen-1-ylcarbamoyl]-pyrrolidine-1-carbonyl}-2-methyl-propyl)-carbamic acid methyl ester. MS: 766.6 (M+H)⁺;

((S)-1-{(S)-2-[2-(4-{2-[(S)-1-(S)-2-Methoxycarbonylamino-3-methyl-butyryl)-pyrrolidin-2-yl]-3H-imidazol-4-yl}-phenyl)-quinolin-6-ylcarbamoyl]-pyrrolidine-1-carbonyl}-2-methyl-propyl)-carbamic acid methyl ester. MS: 767 (M+H)'

### Example 3

### HCV Replicon Assay

This assay measures the ability of the compounds of formula I to inhibit HCV RNA replication, and therefore their potential utility for the treatment of HCV infections. The assay utilizes a reporter as a simple readout for intracellular HCV replicon RNA level. The Renilla luciferase gene was introduced into the first open reading frame of a genotype 1b replicon construct NK5.1 (N. Krieger et al., J. Virol. 2001 75(10):4614), immediately after the internal ribosome entry site (IRES) sequence, and fused with the neomycin phosphotransferase (NPTII) gene via a self-cleavage peptide 2A from foot and mouth disease virus (M. D. Ryan & J. Drew, EMBO 1994 13(4):928-933). After in vitro transcription the RNA was electroporated into human hepatoma Huh7 cells, and G418-resistant colonies were isolated and expanded. Stably selected cell line 2209-23 contains replicative HCV subgenomic RNA, and the activity of Renilla luciferase expressed by the replicon reflects its RNA level in the cells. The assay was carried out in duplicate plates, one in opaque white and one in transparent, in order to measure the anti-viral activity and cytotoxicity of a chemical compound in parallel ensuring the observed activity is not due to decreased cell proliferation or due to cell death.

HCV replicon cells (2209-23), which express Renilla luciferase reporter, were cultured in Dulbecco's MEM (Invitrogen cat no. 10569-010) with 5% fetal bovine serum (FBS, Invitrogen cat. no. 10082-147) and plated onto a 96-well plate at 5000 cells per well, and incubated overnight. Twenty-four hours later, different dilutions of chemical compounds in the growth medium were added to the cells, which were then further incubated at 37° C. for three days. At the end of the incubation time, the cells in white plates were harvested and luciferase activity was measured by using the R. luciferase Assay system (Promega cat no. E2820). All the reagents described in the following paragraph were included in the manufacturer's kit, and the manufacturer's instructions were followed for preparations of the reagents. The cells were washed once with 100 μL of phosphate buffered saline (pH 7.0) (PBS) per well and lysed with 20 μl of 1×R. luciferase Assay lysis buffer prior to incubation at room temperature for 20 min. The plate was then inserted into the Centro LB 960 microplate luminometer (Berthold Technologies), and 100 μl of R. luciferase Assay buffer was injected into each well and the signal measured using a 2-second delay, 2-second measurement program. IC₅₀, the concentration of the drug required for reducing replicon level by 50% in relation to the untreated cell control value, can be calculated from the plot of percentage reduction of the luciferase activity vs. drug concentration as described above.

WST-1 reagent from Roche Diagnostic (cat no. 1644807) was used for the cytotoxicity assay. Ten microliter of WST-1 reagent was added to each well of the transparent plates including wells that contain media alone as blanks Cells were then incubated for 2 h at 37° C., and the OD value was measured using the MRX Revelation microtiter plate reader (Lab System) at 450 nm (reference filter at 650 nm). Again CC₅₀, the concentration of the drug required for reducing cell proliferation by 50% in relation to the untreated cell control value, can be calculated from the plot of percentage reduction of the WST-1 value vs. drug concentration as described above.

### Example 4

Pharmaceutical compositions of the subject Compounds for administration via several routes were prepared as described in this Example.

### Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

### Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

### Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

### Parenteral Formulation (D)

| Ingredient | % wt./wt. | |
|---|---|---|
| Active ingredient | 0.25 | g |
| Sodium Chloride | qs to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

What is claimed is:

1. A compound of the formula: 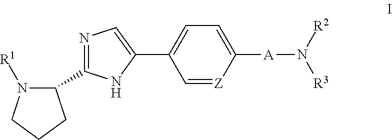
or a pharmaceutically acceptable salt thereof, wherein:
A is a moiety selected from the group consisting of optionally substituted moieties of the formulas: 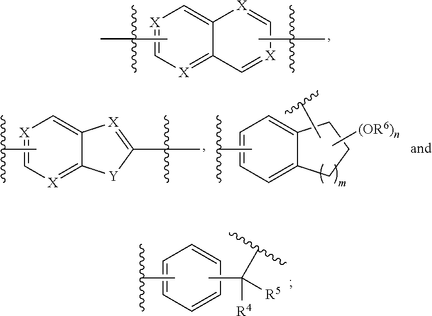
each of which is optionally substituted with C₁₋₆ alkyl; C₁₋₃ haloalkyl, C₁₋₆ alkoxy, halogen, hydroxy, carboxyl, C₁₋₆ alkoxycarbonyl, C₁₋₆ hydroxyalkyl, C₃₋₇ cycloalkyl, cyano or (CH₂)₀₋₃NR^{a}R^{b}.
m is 1 or 2.
n is 0 or 1.
Each X and Z is independently CH or N, provided that no more than two of X are N.
Y is C(═O), O, S, or NR⁷.
R¹ is glycine or an aliphatic amino acid which is optionally is N-acylated with a C₁₋₆ acyl, a benzoyl group or a C₁₋₆ alkoxycarbonyl group.
R² is Pro-R¹ or Ala-R¹;
R³ is hydrogen or C₁₋₆ alkyl.
R⁴ is hydrogen, C₁₋₆ alkyl, C₃₋₆ cycloalkyl, C₁₋₆ haloalkyl, C₁₋₆ hydroxyalkyl, c1-4 alkoxy-C₁₋₆ alkyl, or (CH₂)₀₋₃NR^{a}R^{b}.
R⁵ is hydrogen, C₁₋₆ alkyl, C₁₋₆ haloalkyl, C₁₋₆ hydroxyalkyl, C₁₋₃ alkoxy-C₁₋₆ alkyl, or (CH₂)₀₋₃NR^{a}R^{b}.
R⁶ is hydrogen, C₁₋₆ alkyl, C₁₋₆ haloalkyl, C₁₋₆ hydroxyalkyl, C₁₋₃ alkoxy-C₁₋₆ alkyl, cyano-C₁₋₃ alkyl, or a hydroxy protecting group;
R⁷ is hydrogen, C₁₋₆ alkyl, or nitrogen-protecting group.
R^{a} and R^{b} are independently hydrogen or C₁₋₆ alkyl.

2. The compound according to claim 1, wherein R¹ and R² comprise (L)-proline, (L)-valine and the (L)-aliphatic amino acid fragments.

3. The compound according to claim 2, wherein R¹ is an optionally protected (L)-valine residue.

4. The compound according to claim 2 wherein R² is (L)-Pro-R¹.

5. The compound according to claim 1, wherein R³ is hydrogen.

6. The compound according to claim 1, wherein Z is CH.

7. The compound according to claim 1, wherein A is a moiety selected from the group consisting of: 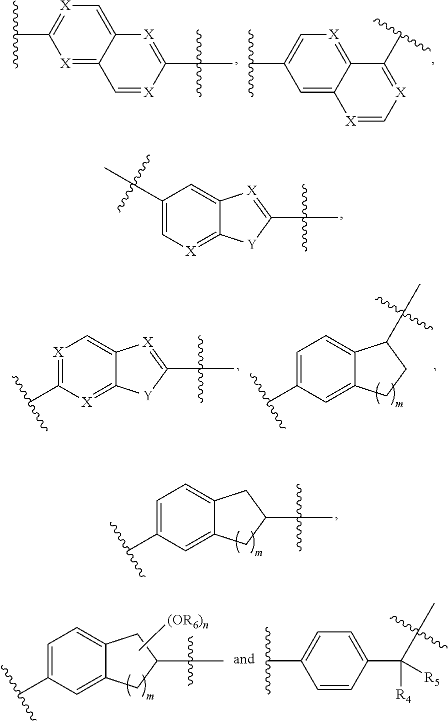
wherein X, Y, R⁴, R⁵, R⁶, m, and n are those defined in claim 1.

8. The compound according to claim 7 wherein A is a moiety selected from the group consisting of: 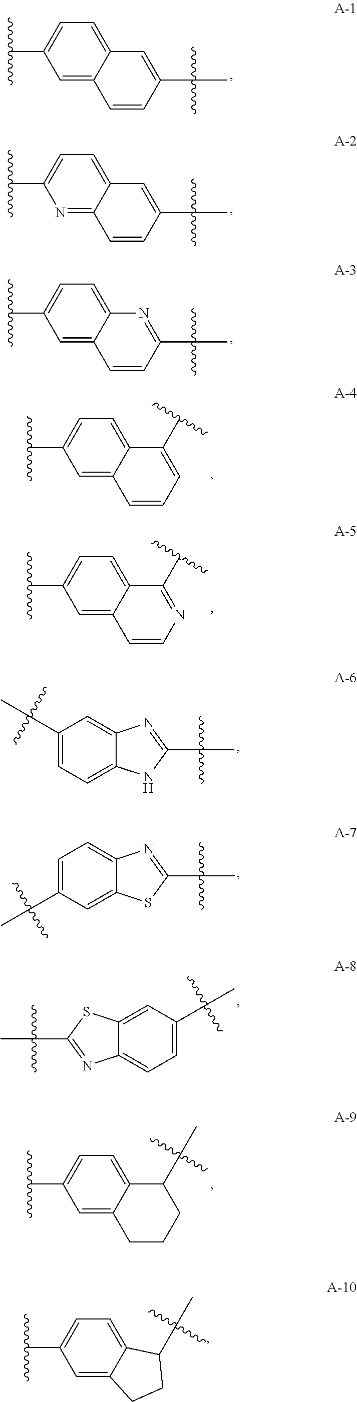 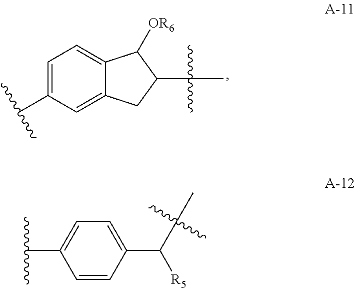
wherein, R⁵, and R⁶ are those defined in claim 1.

9. A method for treating a Hepatitis C Virus (HCV) infection comprising administering to a patient in need of such a treatment a therapeutically effective amount of a compound of claim 1.

10. The method of claim 9 further comprising administering an immune system modulator, an antiviral agent that inhibits replication of HCV, or a combination thereof.

11. The method of claim 10, wherein the immune system modulator is an interferon or chemically derivatized interferon.

12. The method of claim 10, wherein the antiviral agent is selected from the group consisting of a HCV protease inhibitor, another HCV polymerase inhibitor, a HCV helicase inhibitor, a HCV primase inhibitor, a HCV fusion inhibitor, and a combination thereof.

13. A method for inhibiting replication of HCV in a cell comprising administering a compound of claim 1 to the cell.

14. A composition comprising a compound of claim 1 and a pharmaceutically acceptable excipient.
